(19) United States
(12) Patent Application Publication
(10) Pub. No.: US 2010/0094027 A1
(43) Pub. Date: Apr. 15, 2010

(54) Process for the Preparation of Glycerol Formal

(76) Inventors: Todd Coleman, Batesville, AR (US); Allen Blankenship, Batesville, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/544,785

(22) Filed: Aug. 20, 2009

Related U.S. Application Data
(60) Provisional application No. 61/090,281, filed on Aug. 20, 2008.

(51) Int. Cl. C07D 315/00

(52) U.S. Cl. 549/416

(57) ABSTRACT

A process for the preparation of glycerol formal, from a paraformaldehyde and crude glycerin in a condensation reaction without the use of a secondary distilling agent for the removal of the water.

### CROSS REFERENCE TO RELATED APPLICATION(S)

This application claims benefit of U.S. Provisional Patent Application Ser. No. 61/090,281 filed Aug. 20, 2008, the entire disclosure of which is herein incorporated by reference.

### BACKGROUND

1. Field of the Invention

This disclosure relates to the field of processes for the creation of glycerol formal. In particular, to the process of creating glycerol formal from paraformaldehyde and crude glycerin.

2. Description of the Related Art

A condensation reaction is a chemical reaction in which two molecules or moieties (functional groups) combine to form one single molecule, together with the loss of a small molecule. When this small molecule is water, the reaction is known to those skilled in the art as a dehydration reaction.

Examples of condensation reactions known to those skilled in the art include, but are not limited to, esterfication of organic acids, preparation of amides from an amine and an organic acid, and preparation of acetals/ketals from aldehydes/ketones and diols. These reactions are typically catalyzed by a strong acid, such as sulfuric acid, or a strongly-acidic ion-exchange resin.

Condensation reactions are equilibrium reactions (i.e., two opposing reactions occurring simultaneously at the same rate, so that the concentration of each reactant and product remains constant). Those skilled in the art, however, know that a higher conversion of product can be obtained by shifting the equilibrium by the removal of water. This is typically done by using an azeotropic distilling agent such as heptane, benzene, or toluene and a water trap such as a Dean-Stark trap. Another method to remove water, known to those skilled in the art, is by distillation under vacuum without the use of a distillation aid or water trap.

Generally, condensation reactions are used as the basis for making many important polymers. Examples of such polymers include, but are not limited to, nylon, polyester and other condensation polymers and various epoxies.

Paraformaldehyde is the smallest polyoxymethylene. Further, it is the condensation product of formaldehyde with a typical degree of polymerization generally around 8-100 units.

Glycerin is a colorless, odorless, and viscous liquid that is widely used in pharmaceutical formulations. Glycerin has three hydrophilic hydroxyl groups that are generally responsible for its solubility in water and it hygroscopic nature. This particular substructure is a central component of many lipids. In fact, since glycerin generally forms the backbone of triglycerides, it is produced during saponification processes (such as soap making) and transeterfication processes (such as biodiesel production). Thus, glycerin is a common by-product of biodiesel production (via the transesterfication of vegetable oils or animal fats).

As use of and the production of biofuels increases as the demands for replacements for traditional petroleum fuels gain funding and clout in the “green revolution,” the amount of the crude glycerin by-product of these reactions will only increase. Historically, disposal of the crude glycerin by-product of biodiesel production has been by incineration; the by-product has not historically been used as a raw material for secondary reactions. As such, processes that utilize crude glycerin in an efficient and cost-effective manner to create value-added molecules from the crude glycerin by-product of biodiesel production would be valuable and resourceful in the emerging green economy.

Although glycerol formal is not readily available on the chemical commercial market, generally processes for the production of glycerol formal, with the removal of the reaction water, are commonly known in the art. Examples of some such known processes include the following. First, Patent No. ES475962 (Spain, Gimeno 1979) describes a process to prepare glycerol formal from pure glycerin and paraformaldehyde by using a packed column and low pressure to remove the water produced from the condensation reaction. Second, Patent RO78145 (Romania, Burghelea, 1982) describes a process to prepare glycerol formal using technical grade glycerin (90%) and 37% formaldehyde with benzene as an aid to remove water. Third, Patent DE196 48 960 (German, BASF, 1996) describes both a continuous and batch process. In the continuous process, an alcohol and excess ketone are heated to reflux. After a period of time, the ketone is allowed to be removed by distillation, with fresh ketone being added to maintain a constant volume. In a batch process, glycerin and excess acetone are allowed to react in the presence of petroleum ether, with water being collected in a trap. In both these examples, the ketone is utilized in a 4-fold excess with respect to the alcohol.

While the above cited references demonstrate that processes for the production of glycerol formal, with the removal of the reaction water, are generally commonly known in the art, there are several distinct problems with the known processes. Generally, all of the known processes utilize an inert distilling agent in order to remove the water in the condensation reaction. This adds to both the cost and complexity of the production process. For example, the processes of the prior art use a distilling agent, such as benzene, to remove the water (this creates a complex product purification process) and a packed distillation column and vacuum source are required (this increases the equipment costs of the production process). This complexity of the purification process and high cost make the processes of the prior art difficult to manufacture.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein are, among other things, processes for the preparation glycerol formal without the use of a secondary distilling agent to remove the water, in one embodiment from paraformaldehyde and PM 30338 (crude glycerin) with a distillate residue recycle.

In one embodiment, the method is comprised of the steps of: (1) reacting paraformaldehyde and crude glycerin in a condensation reaction without the use of a secondary distilling agent for the removal of water. This method can also be performed with a distillate residue recycle.

Also provided in the present disclosure, is a glycerol formal formed by the process of: (1) providing a paraformaldehyde and a crude glycerin; (2) reacting said paraformaldehyde and said crude glycerin in a condensation reaction without the use of a secondary distilling agent for the removal of water; and (3) segregating said glycerol formal. It is also contemplated that this process for the formation of glycerol formal can be performed with a distillate residue recycle.

Also disclosed herein is a method for the production of glycerol formal, without a distillate residue recycle, the method comprising the steps of: (1) charging crude glycerin, a condensation reaction catalyst, and paraformaldehyde together to create a mixture; (2) heating the mixture to a temperature at which the paraformaldehyde will dissolve; (3) holding the temperature of the mixture until all of the paraformaldehyde is dissolved; (4) holding the temperature of the mixture for another two hours after all of the paraformaldehyde has dissolved; (5) cooling the mixture; (6) neutralizing the mixture; (7) attaching a fractioning column to the mixture; (8) reducing the pressure of the mixture for a first time; (8) heating the mixture to a temperature to remove water; (9) reducing the pressure of the mixture for a second time; (10) increasing the temperature of the mixture and maintaining the pressure of the mixture to collect a first product cut; and (11) increasing the temperature of the mixture and maintaining the temperature of the mixture to collect a second product cut.

In am embodiment of this method, 270.5 grams of crude glycerin are charged in the step of charging.

In another embodiment of this method, 0.5-ml of sulfuric acid are charged as said condensation reaction catalyst in the step of charging.

In yet another embodiment of this method, 60 grams of paraformaldehyde are charged in the step of charging.

In yet another embodiment of this method, the mixture is heated to a temperature of about 100° C. in the step of heating the mixture to a temperature at which the paraformaldehyde will dissolve.

In yet another embodiment of this method, the mixture is held at a temperature of about 100° C. in the step of holding the temperature of the mixture for another two hours after all of the paraformaldehyde has dissolved.

In yet another embodiment of this method, the mixture is cooled to less than 50° C. in the step of cooling the mixture.

In yet another embodiment of this method, the mixture is neutralized by adding about 1.0 ml of 50% caustic.

In still yet another embodiment of this method, the method further comprises the step of adding boiling agents to the mixture after the step of neutralizing the mixture.

In yet another embodiment of this method, the fractioning column is a 15″ Vigreux column.

In still yet another embodiment of this method, the mixture is reduced to a pressure of around 100 mm Hg in the step of reducing the pressure of the mixture for a first time.

In yet another embodiment of this method, the mixture is heated to a temperature of 100° C. in the step of heating the mixture to a temperature to remove water.

In yet another embodiment of this method, the mixture is reduced to a pressure of about 10-20 mm Hg in the step of reducing the pressure of the mixture for a second time.

In still yet another embodiment of this method, the temperature is increased to about 125° C. while maintaining a pressure of about 10-20 mm Hg in the step of increasing the temperature of the mixture and maintaining the pressure of the mixture to collect a first product cut.

In yet another embodiment of this method, the temperature is increased to about 140° C. while maintaining a pressure of about 10-20 mm Hg in the step of increasing the temperature of the mixture and maintaining said pressure of the mixture to collect a second product cut.

Also disclosed herein is a method for the production of glycerol formal with a distillate residue recycle, the method comprising the steps of: (1) charging distillate residue, crude glycerin, a condensation reaction catalyst, and paraformaldehyde together to create a mixture; (2) heating the mixture to a temperature at which the paraformaldehyde will dissolve; (3) holding the temperature of the mixture until all of the paraformaldehyde is dissolved; (4) holding the temperature of the mixture for another two hours after all of the paraformaldehyde has dissolved; (5) cooling the mixture; (6) neutralizing the mixture; (7) attaching a fractioning column to the mixture; (8) reducing the pressure of the mixture; (9) heating the mixture to a temperature to remove water; (10) reducing the pressure of the mixture; (11) increasing the temperature of the mixture and maintaining the pressure of the mixture to collect a first product cut; (12) increasing the temperature of the mixture and maintaining the temperature of the mixture to collect a second product cut; and (13) saving the crude mixture reside for recycling to the next batch.

### BRIEF DESCRIPTION OF THE DRAWINGS

FIG. 1 provides an embodiment of a flowchart of a process for the preparation of glycerol formal and provides molecular diagrams of the molecules.

FIG. 2 provides an embodiment of a flow chart of the process for the preparation of glycerol formal from paraformaldehyde and crude glycerin.

FIG. 3 provides an embodiment of a flow chart of an exemplary step-by-step bench process for the preparation of glycerol formal from paraformaldehyde and crude glycerin, without a distillate residue recycle.

FIG. 4 provides an embodiment of a flow chart of an exemplary step-by-step bench process for the preparation of glycerol formal from paraformaldehyde and crude glycerin, with a distillate residue recycle.

FIG. 5 provides an embodiment of a chart of the raw materials needed in the preparation of glycerol formal, in the process of FIG. 1.

### DESCRIPTION OF THE PREFERRED EMBODIMENT(S)

The following detailed description illustrates by way of example and not by way of limitation. Described herein, among other things, is a new process for the preparation glycerol formal, from paraformaldehyde and crude glycerin, in one embodiment with a distillate residue recycle.

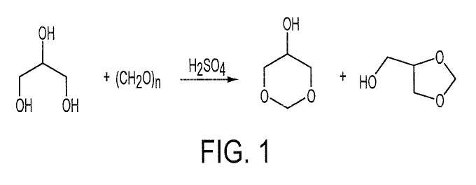

This process, in its simplified form, comprises: using a condensation reaction with the raw materials of paraformaldehyde and crude glycerin, and not using a secondary distilling agent for the removal of water, to produce glycerol formal. One embodiment of this process for the preparation of glycerol formal is shown in the process molecular diagram flow chart of FIG. 1.

Before the process of this disclosure is more fully described herein, it is important to note that additional steps may be performed in certain embodiments, for example in one embodiment the disclosed process will be performed without a distillate residue recycle whereas in another embodiment the disclosed process will be performed with a distillate residue recycle.

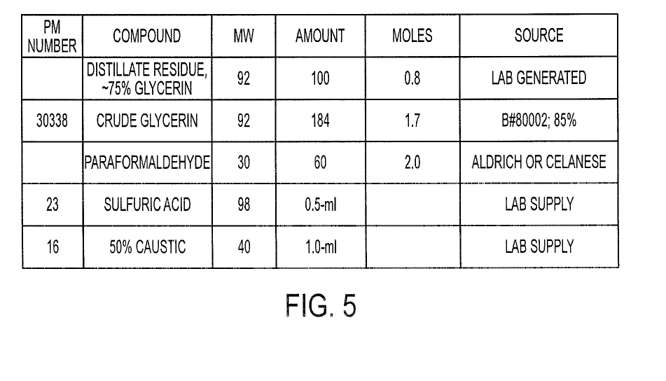

FIG. 5 provides a table of an embodiment of the raw materials used in the preparation of glycerol formal from crude glycerin and paraformaldehyde. It is important to note that is contemplated that any comparable, analogous or sufficient strong acid or strongly-acidic ion-exchange resin known to those of skill in the art now or in the future to catalyze a condensation reaction may be used in place of sulfuric acid. Further, any caustic or other neutralization method or process known to those of skill in the art now or in the future that can be used to neutralize the batch may be used in place of 50% caustic. Identification of these particular chemicals in the chart of FIG. 5 is in no way determinative. Further, the disclosed MW, amounts, and moles are not determinative, and any MW, amounts or moles known to those of skill in the art that would effectively function in the disclosed processes are contemplated.

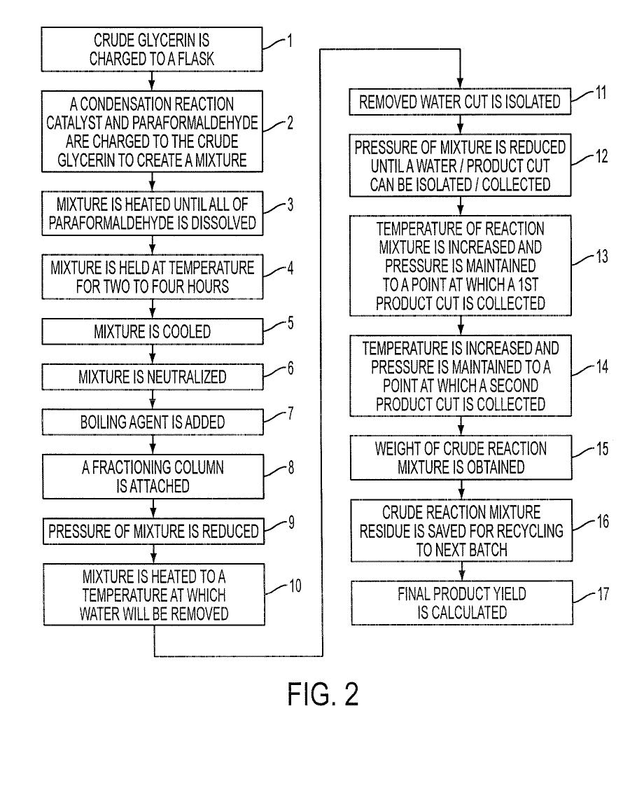

An embodiment of the disclosed process for the preparation glycerol formal, from paraformaldehyde and crude glycerin is shown in the flow chart of FIG. 2. As a preliminary matter, it is noted that at any point in this process a sample of the mixture may be taken and submitted for testing or procedures known to those of skill in the art to have utility in such a reaction. Examples of such tests and/or procedures include, but are not limited to, gas-liquid chromatography analysis, KF water titration, and formaldehyde testing.

In the first step (1) of this embodiment of the disclosed process, crude glycerin is charged to a flask (or similar reaction container/equipment known to those of skill in the art). The amount of crude glycerin charged in this first step is dependant upon whether or not it is the first batch of the series.

Next, in step (2), a condensation reaction catalyst known to those of skill in the art and paraformaldehyde is charged to the crude glycerin to create a mixture. In one embodiment of the disclosed process, the condensation reaction catalyst utilized is sulfuric acid.

Then, in step (3), the mixture is heated until generally all of the paraformaldehyde is dissolved. One embodiment of the process disclosed in FIG. 2, in this step, the time required to reach the point at which all of the paraformaldehyde had dissolved from the mixture is recorded.

After all of the paraformaldehyde is dissolved, in step (4), the crude reaction mixture is held for around two hours at a temperature higher than room temperature.

Next, in step (5), the crude reaction mixture is cooled.

Post-cooling, the crude reaction mixture is neutralized in step (6) by a neutralization method or agent known to those of skill in the art. In one embodiment of the disclosed process, the crude reaction mixture is neutralized by adding a 50% caustic.

Next, in step (7), a boiling agent known to those of skill in the art is added to the mixture. Generally, any boiling agent known to those of skill in the art is contemplated in this disclosure. In one embodiment of the disclosed process of FIG. 2, the boiling agent utilized is Teflon® boiling chips. However, it should be noted that this step is not required and the process of FIG. 2 can be performed without inclusion of this step.

After addition of the boiling agent, a fractioning column or condenser known to those of skill in the art is attached in step (8). In one embodiment of the process of FIG. 2, the fractioning column or condenser utilized is a 15″ Vigreux column.

After column attachment, in step (9) the pressure of the crude reaction mixture is reduced.

After reducing the pressure, in step (10), the crude reaction mixture is generally heated to a temperature at which water will be removed.

Then, in step (11), the removed water cut from the crude reaction mixture is isolated. In an embodiment of this step, the weight of the removed water cut is also recorded.

Next, in step (12), the pressure of the crude reaction mixture is generally reduced until a water/product cut can be collected. In an embodiment of this step, after collection of the water/product cut, the water/product cut is isolated and the weight is recorded. Further, the sample of the water/product cut is submitted for compound analysis and water titration. Generally, any method of compound analysis (e.g., gas-liquid chromatography), water titration (e.g., KF water titration) known to those of skill in the art are contemplated in this step of the disclosed process.

Then, in step (13), the temperature of the crude reaction mixture is generally increased to a temperature and the pressure is maintained to the point at which a first product cut can be collected. In an embodiment of this step, after the first product cut is collected, the cut is isolated and its weight is recorded. Further, the first product sample is submitted for compound analysis, water titration and formaldehyde testing. Generally, any method of compound analysis (e.g., gas-liquid chromatography), water titration (e.g., KF water titration) or formaldehyde testing known to those of skill in the art are contemplated in this step of the disclosed process.

After the first product cut is collected, in step (14), the temperature of the crude reaction mixture is generally increased and the pressure is maintained to such a temperature and level that a second product cut can be collected. In an embodiment of this step, after the second product cut has been collected, the second cut is isolated and its weight is recorded. Then, the second product sample is submitted for compound analysis, water titration and formaldehyde testing. Generally, any method of compound analysis (e.g., gas-liquid chromatography), water titration (e.g., KF water titration) or formaldehyde testing known to those of skill in the art are contemplated in this step of the disclosed process.

In an embodiment of the disclosed process of FIG. 2, following isolation of the second product cut, the weight of the crude reaction mixture residue is obtained in step (15). In one embodiment, the weight of the crude reaction mixture residue is obtained by weighing the flask, pot or equipment that was utilized minus the weight of the utilized fractioning column.

In an embodiment of the disclosed process of FIG. 2, after obtaining the weight of the crude reaction mixture residue, in step (16) the crude reaction mixture residue (i.e., the excess glycerin) is saved for recycling to the next batch.

Further, in an embodiment of the disclosed process of FIG. 2, in a final step (17), the final product yield is calculated using a calculation method or formula known to those of skill in the art.

The disclosed process of FIG. 2 can be performed either with or without a distillate residue recycle. In the embodiment of the process of FIG. 2 in which the process is performed with a distillate residue recycle, prior to step (1) in which the crude glycerin is charged, distillate residue from the previous batch is charged and the crude glycerin is added thereto.

It is noted that the problems of the prior art (i.e., the complexity of the purification process and high cost) are not problems of the disclosed processes of the present application. In the present procedure, glycerol formal is prepared in good yield and high purity using crude glycerin obtained from biodiesel and paraformaldehyde without the removal of the reaction water of condensation. The fact that the reaction water does not need to be removed from the reaction mixture in order to obtain a good yield is advantageous for several reasons: (1) a distillation aid, such as benzene, to remove the water is not required, thus simplifying the process of purification; and (2) a packed distillation column and vacuum source are not required, thus reducing the burden of equipment costs.

Other advantages of the disclosed processes are the ability to use the crude glycerin by-product of the biodiesel process as a raw material. As noted previously, this is essentially a low cost and abundant raw material. Due to the low cost and abundance of glycerin, the reaction can use an excess of alcohol (glycerin) rather than excess formaldehyde (aldehyde/ketone). This allows for a recycle of the reaction residue to increase product yield from formaldehyde and minimizes the likelihood of the formation of high boiling polymers. This results in a safer and more efficient manufacturing process for the production of glycerol formal than those disclosed in the prior art.

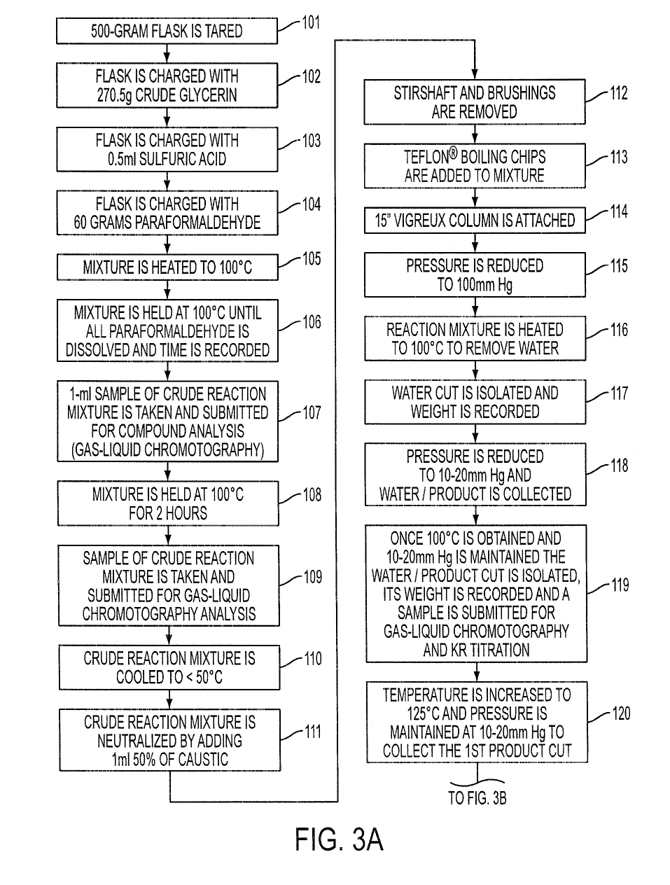

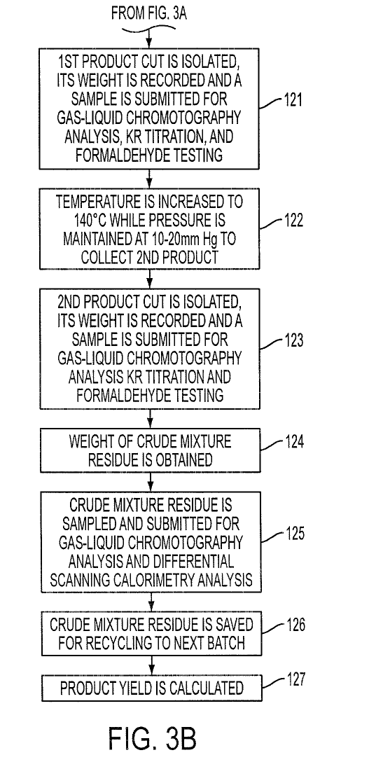

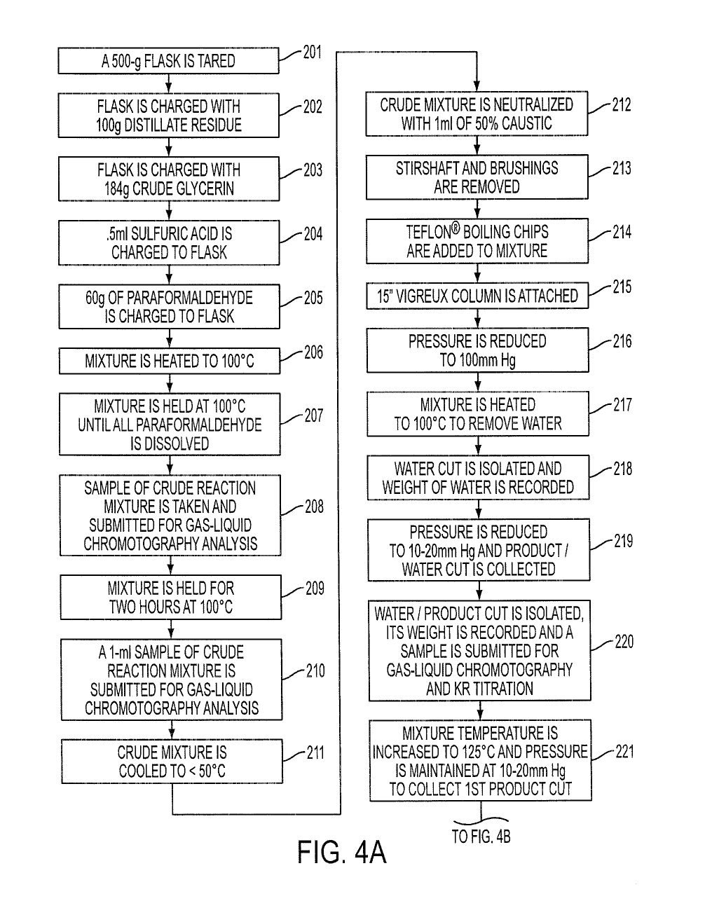

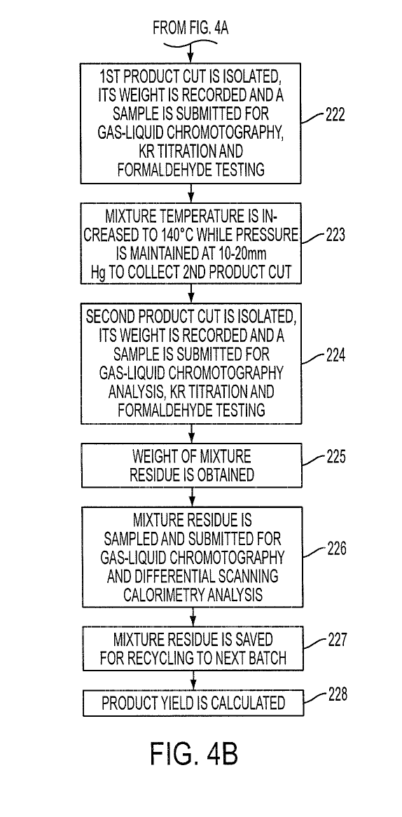

The following examples provide for embodiments of the processes disclosed here-in. The example depicted in FIG. 3 is an exemplary process without a distillate residue recycle. The example depicted in FIG. 4 is an exemplary process with a distillate residue recycle. These processes are generally bench procedures and therefore are exemplary of what may be performed in production. It would be understood by one of ordinary skill in the art that these examples can be adapted to standard commercial operating processes. Further, for the purpose of this disclosure, it is noted that distillation and volume conditions discussed in this embodiment are not determinative, and any functional distillation or volume conditions known to those of skill in the art is contemplated in the processes of this disclosure. Moreover, it is inherent that any specifically identified flask, distillation column or other equipment is not determinative. Any piece of equipment known to those of skill in the art that can properly and effectively function in the given step of the disclosed processes is also contemplated.

### Example 1

To begin, in step (101), a flask is tared. In the embodiment of the process depicted in FIG. 3, the flask is a 500-gram flask.

Then, in step (102), the tared flask is charged with about 270.5 grams of crude glycerin.

Following the charging, in step (103), around 0.5-ml of PM 23 (sulfuric acid) is added to the flask.

Then, in step (104), about 60 grams paraformaldehyde is charged to the reaction flask (6).

After charging the 60 grams of paraformaldehyde, in step (105), the mixture is heated to about 100° C.

In step (106), the mixture is held at about 100° C. until generally all of the parafromaldehyde is dissolved. Step (106) also consists of recording the time required to reach this point (106) at which all of the parafromaldehyde is dissolved.

After recording the time, in step (107), a sample of the crude reaction mixture is taken and then submitted for gas-liquid chromotography analysis using the advance worksheet. In the embodiment of the process depicted in FIG. 3 the sample is a 1-mL sample.

Then, in step (108), the contents of the pot are held for around an additional two hours, generally at 100° C.

Then, in step (109), a sample of the crude reaction mixture is taken and submitted for gas-liquid chromotography analysis using the advance worksheet (109). In the embodiment of the process depicted in FIG. 3 the sample is a 1-mL sample.

After the sample is taken, in step (110), the pot contents are cooled to around <50° C.

Next, in step (111), the batch is neutralized. In this embodiment, the neutralization occurs by adding 1.0-ml of PM 16 (50% caustic) with a plastic pipette. In other embodiments, the batch will be neutralized by other neutralization methods known to those of skill in the art now or in the future.

Post-neutralization, in step (112), the stir shaft and bushings are removed.

Then, after removing the shaft and bushings, in step (113), several Teflon® boiling chips (or comparable boiling chips known to those of skill in the art) are added to the mixture.

Next, in step (114), a 15″ Vigreux column is attached.

After column attachment, in step (115), the pressure is reduced to around 100 mm Hg.

After reducing the pressure, in step (116), the pot is generally heated to around 100° C. to remove water.

Following the step in which the temperature is increased, in step (117), the water cut is isolated and the weight of the water is recorded.

Next, in step (118), the pressure is slowly reduced to generally within the range of 10-20 mm Hg, and the water/product cut is collected.

In step (119), after collection, the water/product cut is isolated and the weight is recorded once the conditions of generally 100° C. and 10-20 mm Hg have been obtained and stabilized. Further, in step (119), the sample of the water/product cut is submitted for gas-liquid chromotography analysis and Karl Fischer water titration.

Then, in step (120), the pot temperature is generally increased to around 125° C., while the pressure is maintained at around 10-20 mm Hg to collect the first product cut.

After increasing the temperature, in step (121), the cut is isolated and the weight is recorded when distillation ceases at around 125° C. and 10-20 mm Hg. In addition, in this step (121), the first product cut sample is submitted gas-liquid chromotography analysis, Karl Fischer water titration, and formaldehyde testing.

In step (122), the pot temperature is generally increased to around 140° C. while the pressure is maintained at around 10-20 mm Hg to collect the second product cut.

Post-collection, in step (123), the second product cut is isolated and the weight is recorded when distillation ceases at around 140° C. and 10-20 mm Hg and the sample is submitted for gas-liquid chromotography analysis, Karl Fischer water titration and formaldehyde testing.

Then, in step (124), the weight of the pot residue is obtained by weighing the pot minus the 15″ Vigreux column.

After obtaining the weight of the pot, in step (125), the pot residue is sampled and submitted for gas-liquid chromotography analysis. Also, a second sample is taken and submitted for differential scanning calorimetry analysis.

In step (126), the pot residue (excess glycerin) is saved for recycling to the next batch.

Finally, in step (127), the yield is calculated using the following equation:

Yield=[(Batch weight×assay)−(Batch weight×% water)]/208.

While the expectant yield of the exemplary process depicted in FIG. 3 varies, in one embodiment it is expected to be between 145 and 185 grams.

### Example 2

To begin, in step (201), a flask is tared. In the embodiment of the process depicted in FIG. 3, the flask is a 500-gram flask.

Then, in step (202), the tared flask is charged with about 100 grams of distillate residue from the previous batch. Generally the typical assay of this distillate is around 75% glycerin.

Then, in step (203), a 500-ml flask is charged with 184 grams of crude glycerin. Generally the typical assay of this glycerin is around 85%.

Following the charging, in step (204), around 0.5-ml of PM 23 (sulfuric acid) is added to the flask.

Then, in step (205), about 60 grams paraformaldehyde is charged to the reaction flask.

After charging the 60 grams of paraformaldehyde, in step (206), the mixture is heated to about 100° C.

In step (207), the mixture is held at about 100° C. until generally all of the paraformaldehyde is dissolved. Step (207) also consists of recording the time required to reach this point (207) at which all of the paraformaldehyde is dissolved.

After recording the time, in step (208), a sample of the crude reaction mixture is taken and then submitted for gas-liquid chromotography analysis using the advance worksheet. In the embodiment of the process depicted in FIG. 3 the sample is a 1-mL sample.

Then, in step (209), the contents of the pot are held for around an additional two hours, generally at 100° C.

Then, in step (210), a sample of the crude reaction mixture is taken and submitted for gas-liquid chromotography analysis using the advance worksheet (210). In the embodiment of the process depicted in FIG. 3 the sample is a 1-mL sample.

After the sample is taken, in step (211), the pot contents are cooled to around <50° C.

Next, in step (212), the batch is neutralized. In this embodiment, the neutralization occurs by adding 1.0-ml of PM 16 (50% caustic) with a plastic pipette. In other embodiments, the batch will be neutralized by other neutralization methods known to those of skill in the art now or in the future.

Post-neutralization, in step (213), the stir shaft and bushings are removed.

Then, after removing the shaft and bushings, in step (214), several Teflon® boiling chips (or comparable boiling chips known to those of skill in the art) are added to the mixture.

Next, in step (215), a 15″ Vigreux column is attached.

After column attachment, in step (216), the pressure is reduced to around 100 mm Hg.

After reducing the pressure, in step (217), the pot is generally heated to around 100° C. to remove water.

Following the step in which the temperature is increased, in step (218), the water cut is isolated and the weight of the water is recorded.

Next, in step (219), the pressure is slowly reduced to generally within the range of 10-20 mm Hg, and the water/product cut is collected.

In step (220), after collection, the water/product cut is isolated and the weight is recorded once the conditions of generally 100° C. and 10-20 mm Hg have been obtained and stabilized. Further, in step (220), the sample of the water/product cut is submitted for gas-liquid chromotography analysis and Karl Fischer water titration.

Then, in step (221), the pot temperature is generally increased to around 125° C., while the pressure is maintained at around 10-20 mm Hg to collect the first product cut.

After increasing the temperature, in step (222), the cut is isolated and the weight is recorded when distillation ceases at around 125° C. and 10-20 mm Hg. In addition, in this step (222), the first product cut sample is submitted gas-liquid chromotography analysis, Karl Fischer water titration, and formaldehyde testing.

In step (223), the pot temperature is generally increased to around 140° C. while the pressure is maintained at around 10-20 mm Hg to collect the second product cut.

Post-collection, in step (224), the second product cut is isolated and the weight is recorded when distillation ceases at around 140° C. and 10-20 mm Hg and the sample is submitted for gas-liquid chromotography analysis, Karl Fischer water titration and formaldehyde testing.

Then, in step (225), the weight of the pot residue is obtained by weighing the pot minus the 15″ Vigreux column.

After obtaining the weight of the pot, in step (226), the pot residue is sampled and submitted for gas-liquid chromotography analysis. Also, a second sample is taken and submitted for differential scanning calorimetry analysis.

In step (227), the pot residue (excess glycerin) is saved for recycling to the next batch.

Finally, in step (228), the yield is calculated using the following equation:

Yield=[(Batch weight×assay)−(Batch weight×% water)]/208.

While the expectant yield of the exemplary process depicted in FIG. 3 varies, in one embodiment it is expected to be between 145 and 185 grams.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A method for the preparation of glycerol formal, the method comprising the steps of:
providing a paraformaldehyde and a crude glycerin; and
reacting said paraformaldehyde and said crude glycerin in a condensation reaction without the use of a secondary distilling agent for the removal of water.

2. The method for the preparation of glycerol formal of claim 1, wherein said condensation reaction is performed with a distillate residue recycle.

3. A glycerol formal formed by the process of:
providing a paraformaldehyde and a crude glycerin;
reacting said paraformaldehyde and said crude glycerin in a condensation reaction without the use of a secondary distilling agent for the removal of water; and
segregating said glycerol formal.

4. The process for the formation of glycerol formal of claim 3, wherein said condensation reaction is performed with a distillate residue recycle.

5. A method for the production of glycerol formal, without a distillate residue recycle, the method comprising the steps of:
charging crude glycerin, a condensation reaction catalyst, and paraformaldehyde together to create a mixture;
heating said mixture to a temperature at which said paraformaldehyde will dissolve;
holding said temperature of said mixture until all of said paraformaldehyde is dissolved;
holding said temperature of said mixture for 2 to 4 hours after all of said paraformaldehyde has dissolved;
cooling said mixture;
neutralizing said mixture;
attaching a fractioning column to said mixture;
reducing the pressure of said mixture for a first time;
heating said mixture to a temperature to remove water;
reducing the pressure of said mixture for a second time;
increasing said temperature of said mixture and maintaining said pressure of said mixture to collect a first product cut; and
increasing said temperature of said mixture and maintaining said temperature of said mixture to collect a second product cut.

6. The method of claim 5, wherein 270.5 grams of crude glycerin are charged in said step of charging.

7. The method of claim 5, wherein 0.5-ml of sulfuric acid are charged as said condensation reaction catalyst in said step of charging.

8. The method of claim 5, wherein 60 grams of paraformaldehyde are charged in said step of charging.

9. The method of claim 5, wherein said mixture is heated to a temperature of about 100° C. in said step of heating said mixture to a temperature at which said paraformaldehyde will dissolve.

10. The method of claim 5, wherein said mixture is held at a temperature of about 100° C. in said step of holding said temperature of said mixture for another two hours after all of said paraformaldehyde has dissolved.

11. The method of claim 5, wherein said mixture is cooled to less than 50° C. in said step of cooling said mixture.

12. The method of claim 5, wherein said mixture is neutralized by adding about 1.0 ml of 50% caustic.

13. The method of claim 5, further comprising the step of adding boiling agents to said mixture after the step of neutralizing said mixture.

14. The method of claim 5, wherein said fractioning column is a 15″ Vigreux column.

15. The method of claim 5, wherein said mixture is reduced to a pressure of around 100 mm Hg in said step of reducing said pressure of said mixture for a first time.

16. The method of claim 5, wherein said mixture is heated to a temperature of 100° C. in said step of heating said mixture to a temperature to remove water.

17. The method of claim 5, wherein said mixture is reduced to a pressure of about 10-20 mm Hg in said step of reducing the pressure of said mixture for a second time.

18. The method of claim 5, wherein said temperature is increased to about 125° C. while maintaining a temperature of about 10-20 mm Hg in said step of increasing said temperature of said mixture and maintaining said pressure of said mixture to collect a first product cut.

19. The method of claim 5, wherein said temperature is increased to about 140° C. while maintaining a temperature of about 10-20 mm Hg in said step of increasing said temperature of said mixture and maintaining said pressure of said mixture to collect a second product cut.

20. A method for the production of glycerol formal with a distillate residue recycle, the method comprising the steps of:
charging distillate residue, crude glycerin, a condensation reaction catalyst, and paraformaldehyde together to create a mixture;
heating said mixture to a temperature at which the paraformaldehyde will dissolve;
holding said temperature of said mixture until all of said paraformaldehyde is dissolved;
holding said temperature of said mixture for another two hours after all of said paraformaldehyde has dissolved;
cooling said mixture;
neutralizing said mixture;
attaching a fractioning column to said mixture;
reducing the pressure of said mixture;
heating said mixture to a temperature to remove water;
reducing the pressure of said mixture;
increasing said temperature of said mixture and maintaining said pressure of said mixture to collect a first product cut;
increasing said temperature of said mixture and maintaining said temperature of said mixture to collect a second product cut; and
saving the crude mixture reside for recycling to the next batch.
